(19) United States
(12) Patent Application Publication
(10) Pub. No.: US 2022/0076694 A1
(43) Pub. Date: Mar. 10, 2022

(54) COGNITIVE IMPAIRMENT DETECTED THROUGH AUDIO RECORDINGS

(71) Applicant: Lifeline Systems Company, Framingham, MA (US)

(72) Inventor: Marten Jeroen PIJL, Eindhoven (NL)

(73) Assignee: Lifeline Systems Company, Framingham, MA (US)

(21) Appl. No.: 17/446,847

(22) Filed: Sep. 3, 2021

Related U.S. Application Data
(60) Provisional application No. 63/075,488, filed on Sep. 8, 2020.

(51) Int. Cl. G10L 25/66; G10L 15/10; G10L 15/18

(52) U.S. Cl. CPC G10L 25/66; G10L 15/1815; G10L 15/10

(57) ABSTRACT

A method for detecting cognitive decline of a user includes receiving speech data corresponding to one or more utterances spoken by the user and processing the received speech data to determine if the speech data is sufficient to perform a cognitive decline analysis of the user. When the speech data is insufficient to perform the cognitive decline analysis, the method also includes initiating a follow-up interaction with the user to collect additional speech data from the user, receiving the additional speech data from the user, and processing the received speech data and the additional speech data to determine a cognitive decline score for the user. The additional speech data corresponds to one or more additional utterances spoken by the user. The method also includes performing an action based on the cognitive decline score of the user.

### CROSS REFERENCE TO RELATED APPLICATIONS

This U.S. patent application claims priority under 35 U.S.C. § 119(e) to U.S. Provisional Application 63/075,488, filed on Sep. 8, 2020. The disclosure of this prior application is considered part of the disclosure of this application and is hereby incorporated by reference in its entirety.

### TECHNICAL FIELD

This disclosure relates to detecting cognitive impairment through audio recordings.

### BACKGROUND

Various user devices are available that connect the user to a service center upon pressing a help button on the user device. Pressing the help button will start an audio connection to the service center. Through monitoring the audio or speech of a subscriber, cognitive decline can potentially be detected at an early stage.

### SUMMARY

One aspect of the disclosure provides a computer-implemented method for detecting cognitive decline of a user. The computer-implemented method when executed on data processing hardware causes the data processing hardware to perform operations that include receiving speech data corresponding to one or more utterances spoken by the user and processing the received speech data to determine if the speech data is sufficient to perform a cognitive decline analysis of the user. When the speech data is insufficient to perform the cognitive decline analysis of the user, the operations also include: initiating a follow-up interaction with the user to collect additional speech data from the user; receiving the additional speech data from the user after initiating the follow-up interaction; and processing the received speech data and the additional speech data to determine a cognitive decline score for the user. The additional speech data corresponds to one or more additional utterances spoken by the user. The operations also include performing an action based on the cognitive decline score for the user.

Implementations of the disclosure may include one or more of the following optional features. In some implementations, receiving the speech data includes receiving current speech data corresponding to a current utterance spoken by the user. In these implementations, receiving the speech data may further include receiving prior speech data corresponding to one or more previous utterances spoken by the user before the current utterance. Here, each of the one or more previous utterances spoken by the user were spoken less than a predetermined period of time before the current utterance. Moreover, when the speech data is sufficient to perform the cognitive decline analysis on the user, the operations may further include processing the received speech data to determine the cognitive decline score for the user

In some examples, processing the speech data includes: generating, using a speech analysis model configured to receive the speech data as input, a speech sufficiency score; and determining the speech data is insufficient to perform the cognitive decline analysis of the user when the speech sufficiency score fails to satisfy a speech sufficiency score threshold. The one or more utterances spoken by the user may be captured by a user device associated with the user, the data processing hardware may reside on a computing system remote from the user device and in communication with the user device via a network, and receiving the speech data corresponding to the one or more utterances spoken by the user may include receiving the speech data from the user device via the network.

In some implementations, the operations further include receiving user information associated with the user that spoke the one or more utterances corresponding to the received speech data. In these implementations, processing the speech data and the additional speech data to determine the cognitive decline score further includes processing the additional information associated with the user to determine the cognitive decline score. In some additional implementations, processing the speech data and the additional speech data to determine the cognitive decline score includes executing a cognitive decline model configured to: receive, as input, the speech data and the additional speech data; and generate, as output, the cognitive decline score. The operations may further include determining whether cognitive decline of the user is detected based on the cognitive decline score. Here, performing the action based on the cognitive decline score for the user may include contacting one or more contacts associated with the user when the cognitive decline of the user is detected.

Another aspect of the disclosure provides a system detecting cognitive decline of a user. The system includes data processing hardware and memory hardware in communication with the data processing hardware and storing instructions that when executed on the data processing hardware causes the data processing hardware to perform operations that include receiving speech data corresponding to one or more utterances spoken by the user and processing the received speech data to determine if the speech data is sufficient to perform a cognitive decline analysis of the user. When the speech data is insufficient to perform the cognitive decline analysis of the user, the operations also include: initiating a follow-up interaction with the user to collect additional speech data from the user; receiving the additional speech data from the user after initiating the follow-up interaction; and processing the received speech data and the additional speech data to determine a cognitive decline score for the user. The additional speech data corresponds to one or more additional utterances spoken by the user. The operations also include performing an action based on the cognitive decline score for the user

This aspect may include one or more of the following optional features. In some implementations, receiving the speech data includes receiving current speech data corresponding to a current utterance spoken by the user. In these implementations, receiving the speech data may further include receiving prior speech data corresponding to one or more previous utterances spoken by the user before the current utterance. Here, each of the one or more previous utterances spoken by the user were spoken less than a predetermined period of time before the current utterance. Moreover, when the speech data is sufficient to perform the cognitive decline analysis on the user, the operations may further include processing the received speech data to determine the cognitive decline score for the user

In some examples, processing the speech data includes: generating, using a speech analysis model configured to receive the speech data as input, a speech sufficiency score; and determining the speech data is insufficient to perform the cognitive decline analysis of the user when the speech sufficiency score fails to satisfy a speech sufficiency score threshold. The one or more utterances spoken by the user may be captured by a user device associated with the user, the data processing hardware may reside on a computing system remote from the user device and in communication with the user device via a network, and receiving the speech data corresponding to the one or more utterances spoken by the user may include receiving the speech data from the user device via the network.

In some implementations, the operations further include receiving user information associated with the user that spoke the one or more utterances corresponding to the received speech data. In these implementations, processing the speech data and the additional speech data to determine the cognitive decline score further includes processing the additional information associated with the user to determine the cognitive decline score. In some additional implementations, processing the speech data and the additional speech data to determine the cognitive decline score includes executing a cognitive decline model configured to: receive, as input, the speech data and the additional speech data; and generate, as output, the cognitive decline score. The operations may further include determining whether cognitive decline of the user is detected based on the cognitive decline score. Here, performing the action based on the cognitive decline score for the user may include contacting one or more contacts associated with the user when the cognitive decline of the user is detected.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system for detecting cognitive decline of a user.

FIG. 2 is a flowchart of an example arrangement of operations for a method of detecting cognitive decline of a user.

FIG. 3 is a schematic view of an example computing device that may be used to implement the systems and methods described herein.

Like reference symbols in the various drawings indicate like elements.

### DETAILED DESCRIPTION

Various user devices, including wearable and portable user devices, are available that allow a user to make emergency contact with a service center as needed. The user may be a subscriber of personal response services that execute on a computing system provided by the service center in the event of an emergency where the user requires help. In one example, such a wearable user device is a smart watch or pendant. The pendant may connect to a lanyard worn around a user's neck to detect falls or other incidents. Also, the user may press a help button on the smart watch or pendant indicating that the user needs help/assistance. In some examples, the user may speak an invocation phrase indicating that the user needs help/assistance without requiring the user to physically press a help button. The user device may be able to capture and transmit audio signals, such as speech spoken by the user. For instance, the user device may capture speech spoken by the user via a microphone in response to the user pressing the help button or speaking the invocation phrase. Additionally, the user device may connect to the personal response service provided by the service center via a cellular or other type of wireless network connection. Similarly, the user device may connect to an access point (e.g., base station) that initiates a call to the service center. At the service center, a personal response agent (PRA) can assess the user's potential emergency or whether the user requires help/assistance, and thereafter provide any necessary support or assistance if needed. The PRAs are trained in assessing emergency situations, and have several options in assisting the subscribers, including verbal assistance, alerting a neighbor, friend, family member, or caregiver of the user, or if necessary, contacting emergency services.

Often the users that subscribe to personal response services may be elderly, and hence, may have various diseases that affect their cognitive ability such as, without limitation, Alzheimer's disease, Parkinson's disease, other dementia-related diseases, or cognitive declines. Also, these users are more likely to experience cognitive impairment over time. Cognitive impairment has a profound impact on one's life and is one of the main contributors in moving to a skilled nursing facility. While there is currently no known cure, early detection offers better chances of coping with the disease, while monitoring of the condition can prevent potentially dangerous situations from occurring due to unobserved loss of ability on the user's part.

Implementations herein are directed toward leveraging audio recordings of user speech data corresponding to utterances spoken by the user to detect cognitive decline/impairment of the user. A cognitive decline analysis model may be trained to process speech data to extract common indicators for detecting cognitive decline of a user. These common indicators extracted from the user speech may include instances where the user is searching for words, exhibiting a limited vocabulary, or the user's speech is otherwise atypical. Similarly, the length of the utterances spoken by the user may help to detect cognitive decline, as well as the fluency of the speech and the depth of vocabulary. Further, decline in memory or other changes in speech patterns may also be used to detect cognitive decline. Accordingly, aspects of the present disclosure are directed toward a cognitive decline service capable of performing cognitive decline analysis on a user based on user speech data.

The user device is capable of establishing an audio communication session with the personal response service upon pressing the help button on the on the user device or speaking a particular invocation phase, e.g., “Help”. During the audio communication session, speech data corresponding to utterances spoken by the user may be processed in order to detect whether or not the user is exhibiting early stages of cognitive decline. However, a certain amount of speech data from the user is needed to perform cognitive decline analysis in order to accurately detect cognitive decline. As such, two or three sentences spoken by the user during a help request to the personal response service may be insufficient for detecting cognitive decline. In scenarios when the speech data received from the user during the help request is insufficient, implementations herein include the cognitive decline service performing follow-up messaging/assessments to obtain additional speech data and/or other information to assess cognitive decline in the user. For example, if a user reads a specified text, this may be used to detect cognitive decline, but such an approach may in some cases be intrusive to the user. If is user speaks to the PRA long enough, sufficient speech may be available to perform the cognitive decline analysis in order to detect potential cognitive decline at an early stage.

Many elderly users live at home unmonitored, and early signs of dementia or cognitive decline may go unnoticed as a result. Similarly, for many users with cognitive impairments, worsening of their condition may not be detected until a critical event takes place. In addition, their evaluation during incidental check-ups can be ascertained by determining whether the subscriber has a good or a bad day. As such, more continuous monitoring may be beneficial.

While cognitive impairment and decline can be detected from speech at an early stage, many of the interactions with the service center will not be of sufficient length to obtain a complete assessment. In addition, relying solely on such interactions may be insufficient, as there may be considerable time between consecutive calls. Notably, while human PRAs are skilled in assessing a user's potential emergency or whether the user requires help/assistance, and thereafter provide any necessary support or assistance if needed, human PRAs are not necessarily trained to ascertain whether or not the user is exhibiting signs of cognitive decline/impairment based on the user speech data. Accordingly, the cognitive decline system uses a cognitive decline analyzer that may leverage a cognitive decline model trained to detect whether or not a user is experiencing cognitive decline impairment by processing user speech data received as input to the cognitive decline model. As will become apparent, the cognitive decline model allows for signs of cognitive decline to be monitored unobtrusively by analyzing conversations between the user and the personal response service.

Prior to conducting the cognitive decline analysis, the speech analyzer may either conclude that the conversation contains sufficient information to determine whether signs of cognitive decline are present, or that further evidence is needed. If sufficient speech data is present, the recorded speech data may be analyzed for indications of cognitive decline. However, if the speech data is insufficient, the subscriber may be prompted to provide additional speech data. Depending on the situation, the subscriber's preferences and ability, or the amount or nature of additional data required, different types of prompting the user to speak further with the call center may be selected. Examples include either starting a conversation with the subscriber through a chatbot or directly prompting the subscriber to perform an assessment, for example, by reading scripted text or answering questions. In some examples, if current speech data received is deemed insufficient for performing cognitive decline analysis while the user is currently communicating with a PRA responsive to a help request, the PRA is prompted to maintain the dialogue with the user so that additional speech data can be attained for use in performing cognitive decline analysis of the user. In these examples, the PRA may receive instructions for performing the assessment by asking the user to answer specific questions by which the additional speech data is collected when the user provides the answers to the specific questions.

In addition to the collected speech data, the cognitive decline analyzer may use user information such as age, medical conditions, medical tests, previous cognitive decline scores output by the cognitive decline model, or information from previous conversations as an input parameter for determining the cognitive decline score. For example, a prior conversation with the user may show potential signs of cognitive decline, but are not conclusive, so a note may be made in the users file to follow up regarding potential cognitive decline in any further contacts with the user. If signs of cognitive decline are found, a variety of actions may be taken, including alerting caregivers at care providers, offering additional support or services to the subscriber, or relaying the information to medical professionals and/or family members.

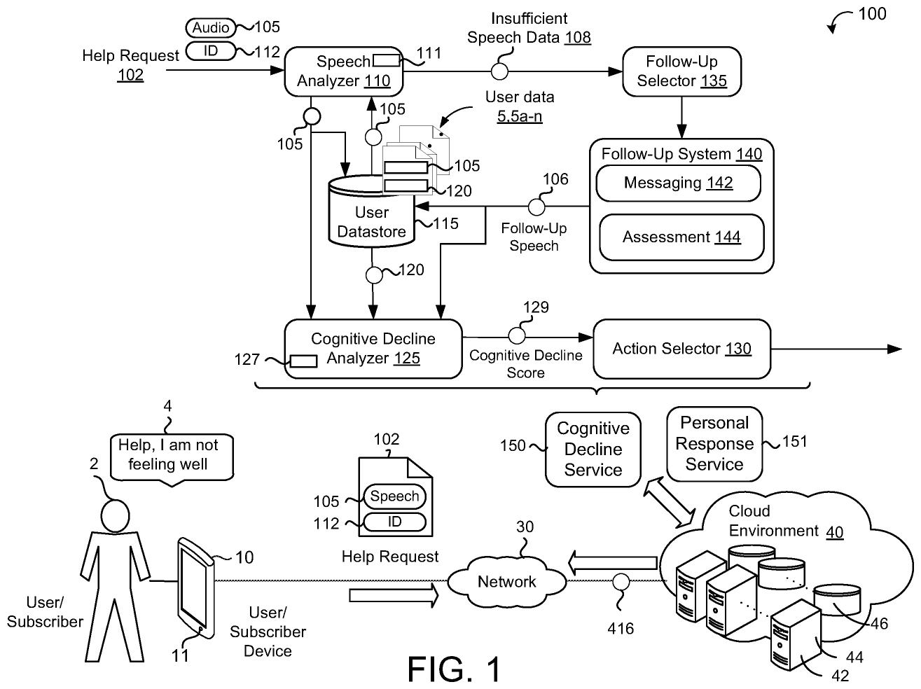

Referring to FIG. 1, in some implementations, a system 100 includes a user/subscriber device 10 associated with a subscriber/user 2 of a personal response service 151, who may communicate, e.g., via a network 30, with a remote system 40. The remote system 40 may be a distributed system (e.g., cloud environment) having scalable/elastic resources 42. The resources 42 include computing resources (e.g., data processing hardware) 44 and/or storage resources (e.g., memory hardware) 46. The user 2 may use the user device 10 to transmit a help request 102 to make emergency contact with the personal response service 151 that executes on the remote system 40 in the event of an emergency where the user 2 requires help. The personal response service 151 may be associated with a service center having personal response agents (PRAs) on call to connect with the user 2 when the help request 102 is received. The user device 10 may correspond to a computing device and/or transceiver device, such as a pendant, a smart watch, a mobile phone, a computer (laptop or desktop), tablet, smart speaker/display, smart appliance, smart headphones, wearable, or vehicle infotainment system. The user device 10 includes or is in communication with one or more microphones for capturing utterances 4 from the user 2.

In the example shown, the user device 10 connects with the personal response service 151 to initiate a help request 102 responsive to the user 2 pressing a button 11 (e.g., a physical button residing on the device 10 or a graphical button displayed on a user interface of the device 10). In other examples, the user 2 may speak a particular invocation phrase (e.g., “Help”) that when detected in streaming audio by the user device 10 causes the user device 10 to connect with the personal response service 151 to initiate the help request 102. Connecting the user device 10 to the personal response service 151 may include connecting the user 2 with the PRA to assess the user's current emergency and provide needed assistance. The user device 10 may connect with the personal response service 151 via a cellular connection or an internet connection. In some examples, the user device 10 connects with the personal responsive service 151 by dialing a phone number associated with the personal response service 151. Upon initiating the help request 102, the user device 10 may commence recording speech data 105 corresponding to an utterance 4 (e.g., “Help, I am not feeling well”) spoken by the user 2 that conveys a current condition, symptoms, or other information explaining why the user 10 requires the personal response service 151 to provide assistance.

The user device 10 transmits the help request 102 to the personal response service 151 via the network 30. The help request 102 includes speech data 105 corresponding to the utterance 4 captured by the user device 10. The help request 102 may additionally include an identifier (ID) 112 that uniquely identifies the particular user/subscriber 2. The ID 112 may include a telephone number associated with the user 2, a sequence of characters assigned to the user 2, and/or token assigned to the user 2 that the personal response service 151 may use to identify the user 2 and look-up any pertinent information 120 associated with the user 2.

The remote system 40 also executes a cognitive decline service 150 that is configured to receive the speech data 105 corresponding to the current utterance 4 provided in the help request 102 transmitted to the personal response service 151 and optionally prior speech data 105 corresponding to one or more previous utterances spoken by the user 2. Specifically, the cognitive decline service 150 is configured to process the received speech data 105 to detect whether or not the user 2 is experiencing cognitive decline/impairment. While examples herein depict the cognitive decline service 150 executing on the remote system 40, the cognitive decline service 150 may alternatively execute solely on the user device 2 or across a combination of the user device 2 and the remote system 40.

The cognitive decline service 150 includes a speech analyzer 110, a cognitive decline analyzer 125, an action selector 130, and a follow-up system 140. The cognitive decline service 150 may include, or access, a user datastore 115 that stores user data sets 5, 5a-n each containing speech data 105 and user information 120 associated with a respective user/subscriber 2 of the personal responsive service 151. The speech data 105 may include prior speech data 105 corresponding to previous utterances spoken by the user 10 during past communications with the personal response service 151 and/or the cognitive decline service 150. For instance, the previous speech data 105 may include a call log of utterances recorded during previous help requests 102 sent to the personal response service 151 where the respective user 10 was in need of help. The prior speech data 105 may be timestamped to indicate when the corresponding utterances were recorded.

The speech analyzer 110 receives the speech data 105 corresponding to the current utterance 4 spoken by the user 2 during the help request 102. The speech analyzer 110 may also receive speech data 105 corresponding to one or more previous utterances spoken by the user 2 before the current utterance 4. For instance, the speech analyzer 110 may use the identifier 112 contained in the help request 102 that uniquely identifies the user 2 to retrieve prior speech data 105 from the respective user data set 5 stored in the user datastore 115 that corresponds to the one or more previous utterances. The speech analyzer 110 is configured to determine whether the received speech data 105 is sufficient for use by the cognitive decline analyzer 125 to determine/detect signs of cognitive decline. Accordingly, the speech analyzer 110 may only retrieve prior speech data 105 corresponding to previous utterances that were recently collected since older utterances do not reflect a current cognitive ability/state of the user 34. In some examples, the speech analyzer 110 only retrieves prior speech data 105 having timestamps associated with utterances that were spoken less than a predetermined period of time before the current utterance 4.

In the example shown, the speech analyzer 110 processes the speech data 104 to determine if the speech data 105 is sufficient for the cognitive decline analyzer 125 to perform a cognitive decline analysis of the user 10. Factors effecting the sufficiency of the speech data 104 include the audio quality, the length of the corresponding utterances represented by the speech data 104, background noise level, background speech level, and durations of individual segments of the utterances spoken by the user. In some examples, the speech analyzer 110 additionally performs speech recognition on the speech data 104 and determines whether a linguistic complexity of the utterances corresponding to the speech data are sufficient for the cognitive decline analyzer 125 to perform the cognitive decline analysis.

In some implementations, the speech analyzer 110 generates, using a speech analysis model 111 configured to receive the speech data 105, a speech sufficiency score and determines the speech data 105 is insufficient to perform the cognitive decline analysis of the user 2 when the speech sufficiency score fails to satisfy a speech sufficiency score threshold. Otherwise, when the speech sufficiency score output from the speech analysis model 111 satisfies the threshold, the speech data 105 is deemed sufficient for use by the cognitive decline analyzer 125 to perform cognitive decline analysis of the user 2. In these implementations, the speech analysis model 111 may be trained on a corpus of training speech samples labeled as sufficient or insufficient for performing cognitive speech analysis. For instances, positive training speech samples may be associated with sufficient speech data that teaches the speech analysis model 111 to learn to predict that the speech data is sufficient, while negative training speech samples may be associated with insufficient speech data that teaches the speech analysis model 111 to learn to predict that the speech data is insufficient. In additional implementations, the speech analyzer 110 is rule-based where metrics and rules may be used based upon the input data requirements for the speech analyzer 110 and the cognitive decline analyzer 125. For example, the speech analyzer 110 may determine that that speech data 105 corresponding to the utterance 4 in the incoming help request 102 satisfies a specified duration and satisfies a specified audio quality.

When the speech analyzer 110 determines the speech data 105 is insufficient to perform the cognitive decline analysis, the cognitive decline service 150 may initiate a follow-up interaction with the user 2 to collect additional speech data 106, e.g., follow-up speech, from the user 10. In some examples, when the speech data 105 is insufficient, the speech analyzer 110 outputs an insufficient speech data indicator 108 for use by a follow-up selector 135 for instructing the follow-up system 140 to initiate the follow-up interaction with the user 2. In these examples, the speech analyzer 110 may be trained to output an insufficient speech data indicator 108 that indicate a quantity and/or type of additional speech data 106 needed to be collected for use by the cognitive decline analyzer 125 to perform the cognitive decline analysis of the user 2. For instance, the indicator 108 may reflect a score or confidence value indicating how much and/or what types of additional speech data 106 is needed for performing cognitive decline analysis.

The follow-up selector 135 uses the insufficient speech data indicator 108 output from the speech analyzer 110 as well as other user information 120 associated with the user 2 to select the type of follow up interaction to help gather additional information to perform the cognitive decline analysis. The user information 120 may be extracted from the user data set 5 stored for the respective user 2 in the user datastore 115. Among other things, the user information 120 may include user preferences and past history for communicating with the user. The selection process may be rule based or may utilize a machine learning model. In either case, the user preferences and past history may factor into the selection process. For example, if the user previously did not respond well to one follow-up interaction approach, then that approach may be avoided, and if the user responded well to a specific follow-up interaction approach in the past, then that approach may be used again.

In some implementations, when only speech data deemed insufficient is available, the cognitive decline analyzer 125 still performs the cognitive decline analysis by processing the insufficient speech data 105. While this is less likely to provide a high-confidence indication of detecting cognitive decline, a decision can be made to instead wait for additional speech data 106 to be received in subsequent help requests 102 and related interactions between the user 2 and the personal response server 151.

The follow-up selector 135 may instruct the follow-up system 140 to initiate the follow-up interaction for collecting the additional speech data 106 from the user 2. The follow-up system 140 may initiate the follow-up interaction using various unobtrusive options. The follow-up interaction may initiate in real-time while the user 2 is currently communicating with a PRA responsive to the help request 102, whereby the PRA may be prompted to collect the additional speech data 106 from the user 2 during the current communication session. One option uses messaging 142 to contact the subscriber on non-related topics, for example by informing the subscriber that their device's battery is low or sending a message to thank the user for their subscription and asking if the user requires any further assistance. This approach can lead to a conversation that allows additional speech to be collected for cognitive decline analysis. The user 2 may explicitly consent to receive messages 142 from the follow-up system 140, and the consent may be revoked at anytime by the user 2. A chatbot or human PRA may provide the messaging 142 to communicate with the user/subscriber 2.

Another option for initiating the follow-up interaction uses an assessment 144 to directly prompt the user 2 to provide the additional speech data 106 directly for additional information. This assessment 144 may include prompting the user to repeat something specific or as for information related to symptoms mentioned by the user 102 in the current help request 102. The assessment may be chosen based upon a context of the help request 102 or prior help requests made by the user 2. The user 2 may explicitly consent to receive assessments 144 from the follow-up system 140, and the consent may be revoked at anytime by the user 2

Another technique for initiating a follow-up interaction with the user to collect additional speech data 106 may be to access a particular service that reminds a subscriber of appointments they have made or that they have prescription that needs to be refilled. These reminders may start a dialog with the user to collect the additional speech data 106 therefrom. The user 2 may explicitly consent to receive reminders from the service and the consent may be revoked at anytime 144 from the follow-up system 140, and the consent may be revoked at anytime by the user 2.

The follow-up system 140 may receive the additional speech data 106 from the user device 2, wherein the additional speech data 106 includes one or more additional utterances spoken by the user and captured by the user device 2. The follow-up system 140 may store the collected additional speech data 106 in the user data store 115 and/or provide the additional speech data 106 to the cognitive decline analyzer 125. In some scenarios, the speech analyzer 110 further processes the combination of the received speech data 105 and the additional speech data 106 collected by the follow-up system 140 to determine whether the combined speech data 105 and the additional speech data 106 is now sufficient for performing the cognitive decline analysis. Thereafter, the cognitive decline analyzer 125 may perform the cognitive decline analysis on the user 2 by processing the speech data 105 and the additional speech data 106 to determine a cognitive decline score 129.

When the speech analyzer 110 determines the speech data 105 is sufficient, or deemed sufficient when combined with additional speech data 106 collected by the follow-up system 140, the cognitive decline analyzer 125 is configured to perform the cognitive decline analysis of the user 2 to output the cognitive decline score 129 indicating whether or not the user 2 is experiencing cognitive decline/impairment. In some examples, the cognitive decline score 129 output from the cognitive decline analyzer 125 includes a binary value indicating the presence or absence of cognitive decline/impairment. In other examples, the cognitive decline score 129 output from the cognitive decline analyzer 125 includes a value indicating a severity and/or a probability that the user is exhibiting signs of cognitive decline/impairment.

In some implementations, the cognitive decline analyzer 125 also receives other user information 120 associated with the user 2 and processes the other user information 120, the speech data 105, and the additional speech data 106 (if required to provide sufficient speech data) to determine the cognitive decline score 129. The other user information 120 associated with the user 2 may be stored in the respective user data set 5 in the user data store 115 and retrieved by the cognitive decline analyzer 125. The user information 120 may include any pertinent information known/collected for the user 2 that may be helpful to accurately detect whether or not the user 2 is currently experiencing cognitive decline. For instance, the user information 120 may include, without limitation, demographic information, health history, medical conditions, prior test information, user preferences, prior cognitive decline scores 129 generated for the user 2, and/or information from prior interactions with the user. For instance, if the user information 120 indicates that that the user has atypical speech due to a permanent jaw injury suffered from an accident, the cognitive decline analyzer 125 may provide less weight to determinations that the user 2 is pronouncing words poorly since this would be attributed to the user's medical condition and not likely cognitive decline (especially if other indicators extracted from the user speech are not conveying signs of cognitive impairment). Additionally, a current cognitive decline score 129 and one or more prior cognitive decline scores 129 previously determined for the user 2 may be used to determine a rate of cognitive decline of the user 2 over time to reflect how fast the user's cognitive ability is deteriorating and predict future cognitive decline of the user 2. Notably, the follow-up selector 135, the follow-up system 140, and the action selector 130 may also use the user information 120 as input when making decisions. For instance, the user information 120 may indicate emergency contacts associated with the user 2 for the action selector 130 to determine to contact in the event of detecting or suspecting cognitive decline of the user 2.

The cognitive decline analyzer 125 or the action selector 130 determines whether or not cognitive decline of the user 2 is detected based on the cognitive decline score 129. In some examples, cognitive decline is detected when the cognitive decline score 129 satisfies a cognitive decline score threshold. If cognitive decline is detected or suspected, such information may be passed on to one or more emergency contacts associated with the user 2 such as a caregiver, doctor, family member, etc. This could include sending an electronic message to a computing device associated with each emergency contact and/or initiating a voice call.

In additional examples, the cognitive decline score 129 is compared to multiple cognitive decline score thresholds each associated with a respective degree/severity of cognitive decline. In this manner, the cognitive decline score 129 in relation to the thresholds may indicate the urgency of the user's need for attention. This can lead to various suggested courses of action selected by the action selector 130 to perform based upon the user's current living situation and current health condition ascertained from the stored user information 120. For example, if the cognitive decline score 129 satisfies only a mild threshold, the action selector 130 may select to perform an action of scheduling a follow up with a doctor or other medical professional. Such recommendation may be sent to a caregiver, family member, the user's doctor, or even the user as indicated by the user preferences. If the cognitive decline score 129 satisfies a severe threshold to indicate severe cognitive decline considered an emergency, the action selector 130 may cause the service 150 to initiate a 911 call and/or a call to an emergency contact of the user to check on the user. As such, the action selector 130 may select an appropriate action among multiple possible actions to perform based on the severity and/or probability of the cognitive decline/impairment indicated by comparisons between the cognitive decline score 129 and the multiple cognitive decline score thresholds. In some scenarios, when the cognitive decline score 129 indicates cognitive decline of the user 2 is not detected, the action selector 130 simply performs the action of storing the cognitive decline score 129 in the user datastore 115 for future reference by the cognitive decline analyzer 125 when performing another cognitive decline analysis of the user 2 responsive to a subsequent help request 102 made by the user 2.

The cognitive decline analyzer 125 may execute a cognitive decline model 127 configured to receive, as input, the speech data 105 (and additional speech data 106 when applicable), and generate, as output, the cognitive decline score 129. The cognitive decline model 127 may include a machine learning model trained on a set of training samples where each training sample includes: training speech data corresponding to one or more utterances spoken by a respective training speaker; and a ground-truth label indicating whether or not the respective training speaker has cognitive decline/impairment. In some examples, the ground-truth label indicates a severity of cognitive decline for the respective training speaker. The cognitive decline model 127 may be further configured to receive user information 120 associated with the user 2 as input for use in generating the cognitive decline score 129 as output. As such, at least a portion of the training samples used to train the cognitive decline model 127 may additionally include respective training user information associated with the respective training speaker.

The cognitive decline service described herein provides a technological improvement in detecting the cognitive decline in users of a user device that communicated with a service center. The cognitive decline service determines whether the data collected in a call is sufficient to conduct a cognitive decline analysis. This determination may also use data collected in prior calls and interactions with the user. When sufficient speech data has not been collected, then the service may initiate further interaction with the user in order to collect additional speech data to be able to perform the cognitive decline analysis. By collecting the data needed to perform a robust cognitive decline analysis will help in early detection of cognitive decline. Earlier detection leads to earlier treatment where possible, but also leads to addition precautions regarding care of the user.

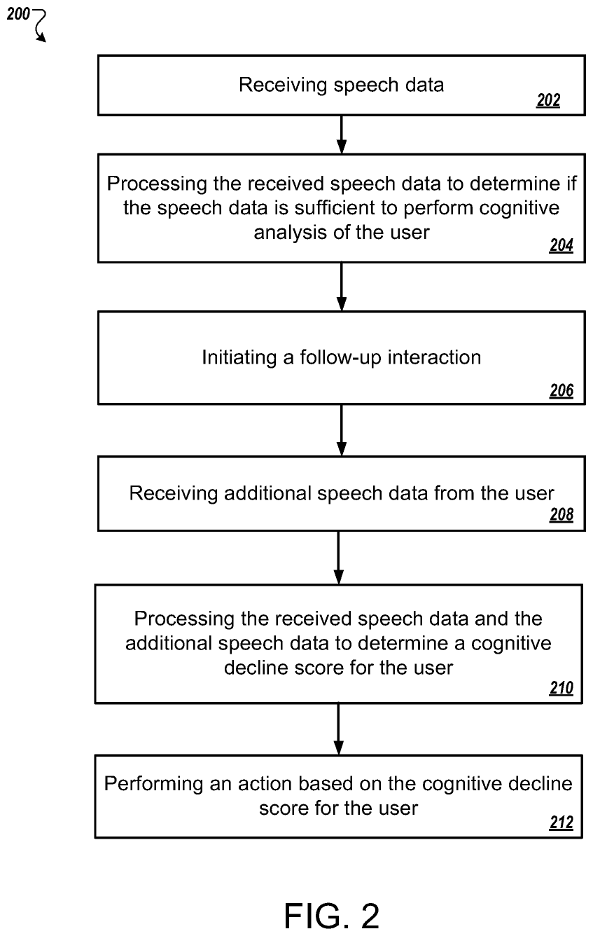

FIG. 2 provides a flowchart for an example arrangement of operations for a method 200 of detecting cognitive decline of a user 2. At operation 202, the method 200 includes receiving speech data 105 corresponding to one or more utterances spoken by the user 2. At operation 204, the method 200 includes processing the received speech data 105 to determine if the speech data 105 is sufficient to perform a cognitive decline analysis of the user 2.

When the speech data 105 is insufficient to perform the cognitive decline analysis on the user, operations 206, 208, 210 are performed. At operation 206, the method 200 includes initiating a follow-up interaction with the user to collect additional speech data 106 from the user. At operation 208, the method 200 includes receiving the additional speech data 106 from the user 2 after initiating the follow-up interaction. Here, the additional speech data corresponds to one or more additional utterances spoken by the user. At operation 210, the method 200 includes processing the received speech data 105 and the additional speech data 106 to determine a cognitive decline score 129 for the user 2.

At operation 212, the method 200 includes performing an action based on the cognitive decline score 129 for the user 2. For instance, the action may include contacting one or more contacts associated with the user based on the cognitive decline score 129.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

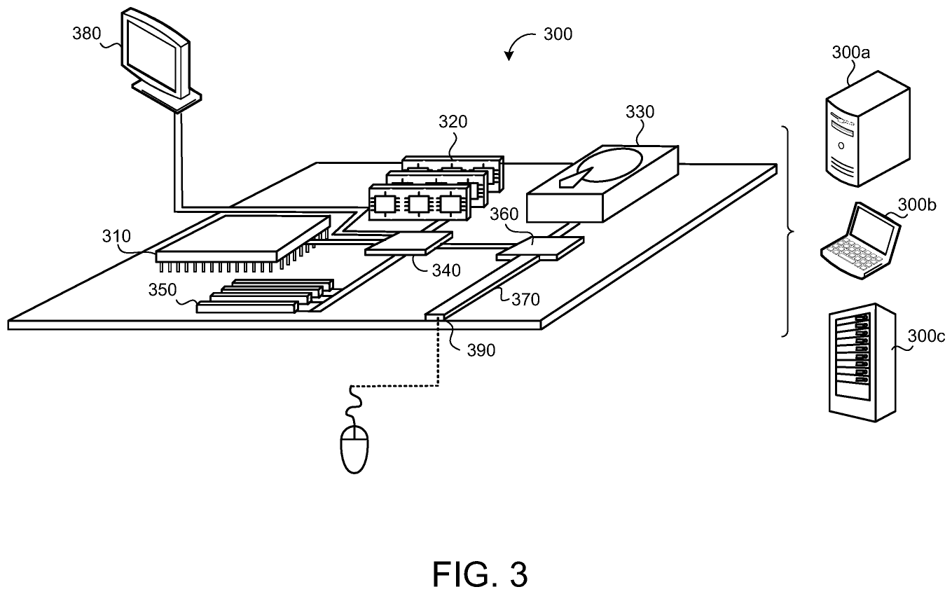

FIG. 3 is schematic view of an example computing device 300 that may be used to implement the systems and methods described in this document. The computing device 300 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 300 includes a processor 310, memory 320, a storage device 330, a high-speed interface/controller 340 connecting to the memory 320 and high-speed expansion ports 350, and a low speed interface/controller 360 connecting to a low speed bus 370 and a storage device 330. Each of the components 310, 320, 330, 340, 350, and 360, are interconnected using various busses, and may be mounted on a common motherboard or in other manners as appropriate. The processor 310 can process instructions for execution within the computing device 300, including instructions stored in the memory 320 or on the storage device 330 to display graphical information for a graphical user interface (GUI) on an external input/output device, such as display 380 coupled to high speed interface 340. In other implementations, multiple processors and/or multiple buses may be used, as appropriate, along with multiple memories and types of memory. Also, multiple computing devices 300 may be connected, with each device providing portions of the necessary operations (e.g., as a server bank, a group of blade servers, or a multi-processor system).

The memory 320 stores information non-transitorily within the computing device 300. The memory 320 may be a computer-readable medium, a volatile memory unit(s), or non-volatile memory unit(s). The non-transitory memory 320 may be physical devices used to store programs (e.g., sequences of instructions) or data (e.g., program state information) on a temporary or permanent basis for use by the computing device 300. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 330 is capable of providing mass storage for the computing device 300. In some implementations, the storage device 330 is a computer-readable medium. In various different implementations, the storage device 330 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 320, the storage device 330, or memory on processor 310.

The high speed controller 340 manages bandwidth-intensive operations for the computing device 300, while the low speed controller 360 manages lower bandwidth-intensive operations. Such allocation of duties is exemplary only. In some implementations, the high-speed controller 340 is coupled to the memory 320, the display 380 (e.g., through a graphics processor or accelerator), and to the high-speed expansion ports 350, which may accept various expansion cards (not shown). In some implementations, the low-speed controller 360 is coupled to the storage device 330 and a low-speed expansion port 390. The low-speed expansion port 390, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 300 may be implemented in a number of different forms, as shown in the figure. For example, it may be implemented as a standard server 300a or multiple times in a group of such servers 300a, as a laptop computer 300b, or as part of a rack server system 300c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims

What is claimed is:

1. A computer-implemented method for detecting cognitive decline of a user, the computer-implemented method when executed on data processing hardware causes the data processing hardware to perform operations comprising:
receiving speech data corresponding to one or more utterances spoken by the user;
processing the received speech data to determine if the speech data is sufficient to perform a cognitive decline analysis of the user;
when the speech data is insufficient to perform the cognitive decline analysis on the user:
initiating a follow-up interaction with the user to collect additional speech data from the user;
receiving the additional speech data from the user after initiating the follow-up interaction, the additional speech data corresponding to one or more additional utterances spoken by the user; and
processing the received speech data and the additional speech data to determine a cognitive decline score for the user; and
performing an action based on the cognitive decline score for the user.

2. The computer-implemented method of claim 1, wherein, when the speech data is sufficient to perform the cognitive decline analysis on the user, the operations further comprise processing the received speech data to determine the cognitive decline score for the user.

3. The computer-implemented method of claim 1, wherein receiving the speech data comprises receiving current speech data corresponding to a current utterance spoken by the user.

4. The computer-implemented method of claim 3, wherein receiving the speech data further comprises receiving prior speech data corresponding to one or more previous utterances spoken by the user before the current utterance.

5. The computer-implemented method of claim 4, wherein each of the one or more previous utterances spoken by the user were spoken less than a predetermined period of time before the current utterance.

6. The computer-implemented method of claim 1, wherein processing the speech data comprises:
generating, using a speech analysis model configured to receive the speech data as input, a speech sufficiency score; and
determining the speech data is insufficient to perform the cognitive decline analysis of the user when the speech sufficiency score fails to satisfy a speech sufficiency score threshold.

7. The computer-implemented method of claim 1, wherein:
the one or more utterances spoken by the user are captured by a user device associated with the user;
the data processing hardware resides on a computing system remote from the user device and in communication with the user device via a network; and
receiving the speech data corresponding to the one or more utterances spoken by the user comprises receiving the speech data from the user device via the network.

8. The computer-implemented method of claim 1, wherein the operations further comprise:
receiving user information associated with the user that spoke the one or more utterances corresponding to the received speech data,
wherein processing the speech data and the additional speech data to determine the cognitive decline score further comprises processing the additional information associated with the user to determine the cognitive decline score.

9. The computer-implemented method of claim 1, wherein processing the speech data and the additional speech data to determine the cognitive decline score comprises executing a cognitive decline model configured to:
receive, as input, the speech data and the additional speech data; and
generate, as output, the cognitive decline score.

10. The computer-implemented method of claim 1, wherein the operations further comprise:
determining whether cognitive decline of the user is detected based on the cognitive decline score,
wherein performing the action based on the cognitive decline score for the user comprises contacting one or more contacts associated with the user when cognitive decline of the user is detected.

11. A system for detecting cognitive decline of a user, the system comprising:
data processing hardware; and
memory hardware in communication with the data processing hardware and storing instructions that when executed on the data processing hardware causes the data processing hardware to perform operations comprising:
receiving speech data corresponding to one or more utterances spoken by the user;
processing the received speech data to determine if the speech data is sufficient to perform a cognitive decline analysis of the user;
when the speech data is insufficient to perform the cognitive decline analysis on the user:
initiating a follow-up interaction with the user to collect additional speech data from the user;
receiving the additional speech data from the user after initiating the follow-up interaction, the additional speech data corresponding to one or more additional utterances spoken by the user; and
processing the received speech data and the additional speech data to determine a cognitive decline score for the user; and
performing an action based on the cognitive decline score for the user.

12. The system of claim 11, wherein, when the speech data is sufficient to perform the cognitive decline analysis on the user, the operations further comprise processing the received speech data to determine the cognitive decline score for the user.

13. The system of claim 11, wherein receiving the speech data comprises receiving current speech data corresponding to a current utterance spoken by the user.

14. The system of claim 13, wherein receiving the speech data further comprises receiving prior speech data corresponding to one or more previous utterances spoken by the user before the current utterance.

15. The system of claim 14, wherein each of the one or more previous utterances spoken by the user were spoken less than a predetermined period of time before the current utterance.

16. The system of claim 11, wherein processing the speech data comprises:
generating, using a speech analysis model configured to receive the speech data as input, a speech sufficiency score; and
determining the speech data is insufficient to perform the cognitive decline analysis of the user when the speech sufficiency score fails to satisfy a speech sufficiency score threshold.

17. The system of claim 11, wherein:
the one or more utterances spoken by the user are captured by a user device associated with the user;
the data processing hardware resides on a computing system remote from the user device and in communication with the user device via a network; and
receiving the speech data corresponding to the one or more utterances spoken by the user comprises receiving the speech data from the user device via the network.

18. The system of claim 11, wherein the operations further comprise:
receiving user information associated with the user that spoke the one or more utterances corresponding to the received speech data,
wherein processing the speech data and the additional speech data to determine the cognitive decline score further comprises processing the additional information associated with the user to determine the cognitive decline score.

19. The system of claim 11, wherein processing the speech data and the additional speech data to determine the cognitive decline score comprises executing a cognitive decline model configured to:
receive, as input, the speech data and the additional speech data; and
generate, as output, the cognitive decline score.

20. The system of claim 11, wherein the operations further comprise:
determining whether cognitive decline of the user is detected based on the cognitive decline score,
wherein performing the action based on the cognitive decline score for the user comprises contacting one or more contacts associated with the user when cognitive decline of the user is detected.
